(19) United States
(12) Patent Application Publication
(10) Pub. No.: US 2010/0113893 A1
(43) Pub. Date: May 6, 2010

(54) METHOD FOR MEASURING PHYSIOLOGICAL STRESS

(75) Inventors: Richard J. Cohen, Chestnut Hill, MA (US); Danilo Scepanovic, Cambridge, MA (US)

Correspondence Address: CHOATE, HALL & STEWART LLP, TWO INTERNATIONAL PLACE, BOSTON, MA, 02110, US

(73) Assignee: Massachusetts Institute of Technology, Cambridge, MA (US)

(21) Appl. No.: 12/445,139

(22) PCT Filed: Oct. 11, 2007
(86) PCT No.: PCT/US2007/081081

§ 371 Date: Dec. 21, 2009

Related U.S. Application Data
(60) Provisional application No. 60/851,241, filed on Oct. 12, 2006.

(51) Int. Cl. A61B 5/02

(52) U.S. Cl. 600/301

(57) ABSTRACT

Method for determining physiological stress. One or more physiologic signals in a subject is detected. The one or more physiologic signals are processed to obtain one or more processed signals. The subject's stress level is estimated from the one or more processed signals. The one or more physiological signals may include the electrocardiogram, instantaneous lung volume or other signal reflecting respiratory activity, or the arterial blood pressure or other signal reflecting cardiovascular activity.

This application claims priority to provisional application Ser. No. 60/851,241 filed Oct. 12, 2006, the contents of which are incorporated herein by reference.

### BACKGROUND OF THE INVENTION

This invention relates to methods for measuring physiological stress and more particularly to such methods involving an assessment of sympathetic and parasympathetic activity.

Physiological stress exists in two varieties: 1) mental, often caused by worry, fear, and anxiety, and 2) physical, in response to exertion, confinement, and general body discomfort. Regardless of the source of stress, it affects the body through the same mechanism, increasing heart rate, cortisol and epinephrine production, allowing us to escape danger or giving us the necessary energy to complete a hard task. Prolonged stress has many negative effects however. It has been linked to mental illness, heart problems, immune system deficiencies, accelerated cell aging, oral health problems, anxiety, depression, and other serious diseases. Acute stress, on the other hand, is generally not harmful, but may be a good indicator of a person's physiologic state. For example, low fetal heart rate variability (a consequence of physiological stress) has been correlated to birthing problems and is routinely measured during childbirth.

Stress can be defined as the dominance of the sympathetic branch of the autonomic nervous system (ANS) over the parasympathetic branch. This is a valid and intuitively pleasing definition because sympathetic activity results in physiological changes that we associate with stressful situations: increases in heart rate, vasoconstriction, etc., and parasympathetic activity has the opposite effect and is generally highest during rest (see FIG. 3).

Thus, to estimate a person's stress level, one needs an estimate of his or her autonomic system function. Furthermore, in order for the stress estimation method to be immediately useful for patient monitoring, these estimates must be obtainable in real time or with a reasonable (at most 30 second) lag. However, stress estimation over longer time periods (minutes or hours) can also be a useful diagnostic, so this invention covers methods for doing both.

Autonomic nervous system function can be measured in a variety of ways. A direct method would involve placing microelectrodes in the vicinity of the vagus and the sympathetic nerves in order to record the electrical activity of both of these autonomic nervous system branches. This method suffers from the highly invasive procedure and difficult implementation, both of which make it impractical for use on human subjects. Sarbach et al. (U.S. Pat. No. 7,049,149) describe an alternative method wherein the subject's stress state is estimated via chemical analysis of his or her exhalation. A similar analysis could be done on the subject's blood, looking for certain hormones that are released by sympathetic activity (cortisol, adrenaline, etc.). Sympathetic and parasympathetic activity can also be estimated by looking at the frequency spectrum of a long sequence of RR intervals, as described in [1]. The numbers in brackets refer to the references appended hereto, the contents of which are incorporated herein by reference.

### SUMMARY OF THE INVENTION

The method of the invention for determining physiological stress involves detecting one or more physiologic signals in a subject, processing the one or more physiologic signals to obtain one or more processed signals, and estimating from the one or more processed signals the subject's stress level. The one or more physiological signals may include the electrocardiogram, or the instantaneous lung volume or other signal reflecting respiratory activity, or the arterial blood pressure or other signal reflecting cardiovascular activity.

One preferred embodiment of the invention involves detecting one or more physiologic signals in a subject and processing the physiologic signal to obtain a sequence of intervals related to time intervals between heartbeats. In a preferred embodiment the subject's stress level is estimated from signals derived from the sequence of intervals. In another preferred embodiment, the sequence of intervals is evaluated in real time. In yet another embodiment, the sequence of intervals is evaluated in a longer time window. The physiologic signal may be an electrocardiogram. A suitable time window is on the order of one to ten seconds.

In a preferred embodiment, the sequence of intervals is the actual inter-beat interval. The processed signal derived from the sequence of intervals may also be the instantaneous rate of change of heart rate. The processed signal derived from the sequence of intervals may also be the second difference of heart rate. In yet another embodiment, the processed signal derived from the sequence of intervals is the mean of the absolute value of the first difference of the intervals within a short window. In yet another embodiment, the processed signal derived from the sequence of intervals is the percent change in the inter-beat interval per unit time. The processed signal derived from the sequence of intervals may also be a linear correlation coefficient of a moderately sized window of inter-beat intervals. A suitable moderately sized window is approximately 20 seconds. The processed signal derived from the sequence of intervals may also be shot noise or spectral curvature.

In a preferred embodiment, the sequence of intervals is used to estimate the magnitude of sympathetic and parasympathetic activity. In this aspect, physiological stress is determined from a function of the sympathetic and parasympathetic activity. In a preferred embodiment, stress is the ratio of sympathetic to parasympathetic activity. In another embodiment, stress is estimated as a linear combination of sympathetic and parasympathetic activity.

### BRIEF DESCRIPTION OF THE DRAWING

FIG. 1A is a graph showing an instantaneous lung volume to heart rate transfer function.

FIG. 1B is a schematic illustration of an entire closed-loop model of circulatory control.

FIGS. 2A-D are waveforms showing an estimate of sympathetic and parasympathetic tone for supine and standing patients.

FIG. 3A is a graph showing the short-window estimate of parasympathetic and sympathetic function for a supine subject.

FIG. 3B is a graph showing the short-window estimate of parasympathetic and sympathetic function for a standing subject.

FIGS. 4A-C are graphs showing an extrapolation procedure that can be used with cardiovascular system identification to improve the performance on limited amounts of data.

FIGS. 5A-D are bar graphs showing sympathetic and parasympathetic estimates for each of eight subjects.

FIG. 6 is a graph of normalized value versus time showing the result of estimating sympathetic and parasympathetic tone on a patient suffering from sleep apnea.

FIGS. 7A-D show population results for the time-domain signal S1.

FIGS. 8A-D show population results for the time-domain signal S2.

FIGS. 9A-D show population results for the time-domain signal S3.

FIGS. 10A-D show population results for the time-domain signal S4.

FIGS. 11A-D show population results for the time-domain signal S5.

FIGS. 12A-D show the population results for the time-domain signal S6.

FIGS. 13A-F show the parasympathetic ratio for standing and supine conditions based on signals S1-S6.

FIG. 14A are graphs showing the whole signal autocorrelations for supine propranolol subjects.

FIG. 14B are graphs showing the whole signal autocorrelations for standing atropine subjects.

FIG. 15 is a schematic illustration showing how shot noise is calculated.

FIGS. 16A-D are graphs showing population results for shot noise.

FIGS. 17A-D are graphs showing population results for spectral curvature.

FIG. 18A is a graph showing the parasympathetic ratio based on spectral curvature.

FIG. 18B is a graph showing the parasympathetic ratio for shot noise.

FIGS. 19A-D are waveforms showing the results of signals S1, S2, S3, and shot noise respectively.

FIG. 20 is a graph showing parameterized instantaneous lung volumes/heart rate transfer function.

### DESCRIPTION OF PREFERRED EMBODIMENT

The method of the invention for determining physiological stress involves detecting one or more physiologic signals in a subject, processing the one or more physiologic signals to obtain one or more processed signals, and estimating from the one or more processed signals the subject's stress level. The one or more physiological signals may include the electrocardiogram, or the instantaneous lung volume or other signal reflecting respiratory activity, or the arterial blood pressure or other signal reflecting cardiovascular activity.

In one preferred embodiment the present invention uses a measure of the heart's electrical activity (for example, from an electrocardiogram or any method that can record the occurrences of heart beats) in order to estimate the desired sympathetic and parasympathetic indices. This approach is valid because the vagus nerve impinges on the sinoatrial node and the sympathetic nerves synapse on the sinoatrial node as well as the surrounding myocardium. The activity of these neurons directly affects the pacemaker function of the sinoatrial node as well as the conductive properties of the surrounding myocardium, thereby effecting changes in the heart's inter-beat intervals. Inter-beat intervals will be referred to as RR intervals, as they are typically calculated by measuring the time between successive QRS complexes on an ECG record. By measuring the timing between contractions of the heart, one can estimate parasympathetic and sympathetic nervous activity. This approach is also more practical than the previous art because it does not explicitly require a specialized apparatus to collect data, and can use, but is not confined to, the standard ECG available in all hospitals.

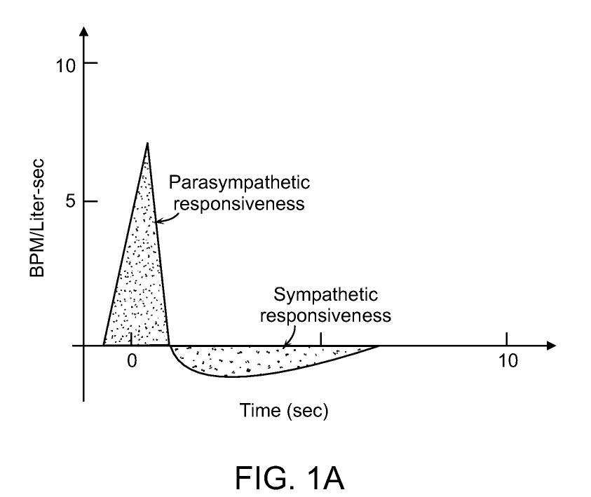

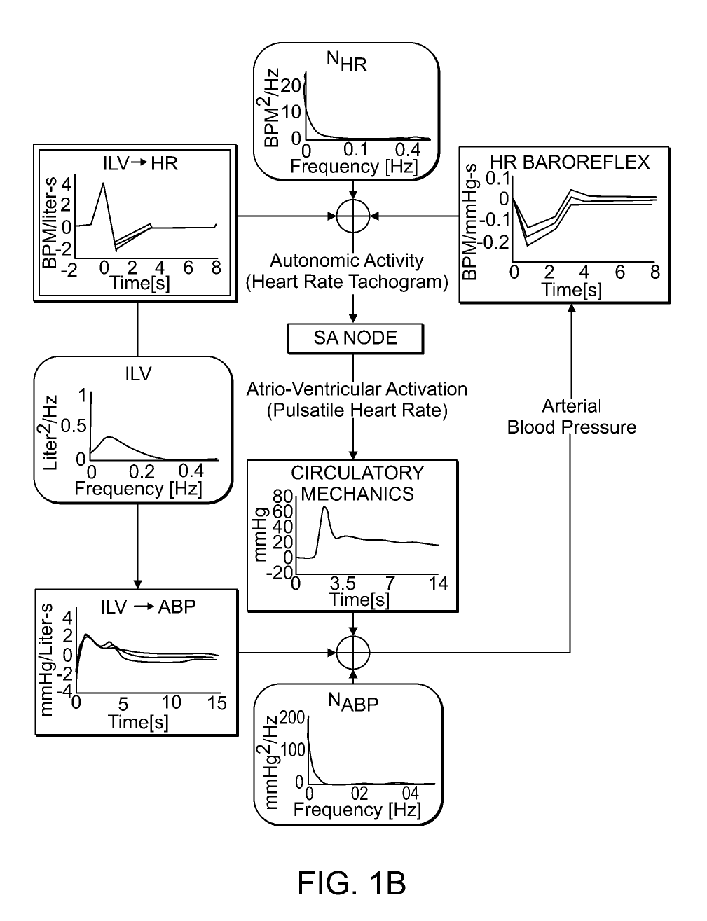

It has been shown that the cardiovascular system can be described by transfer functions that relate instantaneous lung volume, heart rate, and arterial blood pressure in a closed-loop fashion as shown in FIG. 1B [2-5]. It has further been shown that the transfer function relating instantaneous lung volume to changes in heart rate has features that correlate to parasympathetic and sympathetic activation as in FIG. 1A [6]. The transfer functions mentioned above can be obtained by cardiovascular system identification (CSI). As part of the present invention, we include methods for using CSI to extract the desired transfer function and analyze the transfer function further to obtain autonomic indices. Typically, CSI requires about five minutes of data; we describe methods for using CSI on shorter data segments in order to estimate autonomic function with greater time resolution, as well as to improve the overall estimate obtained from the long data record.

In addition to the CSI methods mentioned above, we describe a series of processed signals that can be calculated from the recorded RR intervals (these signals will be referred to as S1-S6, shot noise, and spectral curvature).

The following is a list of processed signals that can be derived from the RR interval sequence, all of which have shown a correlation with parasympathetic or sympathetic dominance:
S1: the RR interval sequence itself
S2: the instantaneous rate of change of the heart rate
S3: the second difference of the heart rate
S4: the mean of the absolute value of the first difference of RR intervals within a short window (about 5 seconds)
S5: the percent change in RR interval per unit time
S6: the linear correlation coefficient of a moderately-sized window (about 20 seconds) of RR intervals
Shot noise: an autocorrelation function is calculated using short RR interval windows (about 5 seconds). The shot noise is the difference between the autocorrelation at lag 0 and the value for lag 0 obtained by extrapolation using a linear (or nonlinear) fit to the autocorrelation values at lags 1 and 2 (or more)
Spectral curvature: a short window of RR intervals is convolved with a time-reversed moderately-sized window of RR intervals, both windows being centered on the same time. A Fourier transform of this special autocorrelation is calculated, and the average spectral energy is computed for three frequency bins, with edges at 0, 0.04, 0.125, and 0.4 Hz. The second difference of these three values yields a single value which is termed the spectral curvature
MAP estimation: the Viterbi algorithm (as described in literature) is used to solve the maximum a posteriori estimate of the ILV-)HR transfer function at arbitrary time points under a Hidden Markov Model

We also describe methods for correlating these signals to the level of sympathetic and parasympathetic activation, as well as to a measure of stress. We also describe a method for estimating autonomic activation on longer stretches of data, using an autocorrelation method. We also describe a Hidden Markov Model approach for quantifying the relationship between instantaneous heart rate and instantaneous lung volume (both obtainable from ECG). By solving for the hidden states by a method such as the maximum a posteriori estimate, we can obtain a time course of sympathetic and parasympathetic activation. We also describe an integrate and fire model of the sinoatrial node that is affected by inputs from the sympathetic and parasympathetic nerves. The model can be solved analytically or via simulation to yield the expected distribution of RR intervals as a function of parasympathetic tone. We also describe a more complicated model of the sinoatrial node that uses differential equations to calculate the change in membrane potential (similar to the Hodgkin-Huxley model) and that includes inputs from sympathetic and parasympathetic nerves. This model can be used to empirically derive the RR interval distributions for various levels of autonomic nervous system function and these distributions can be fit to the observed data to estimate autonomic nervous system function in a particular subject. It may also be possible to relate the results of this model to the electrical activity that is picked up by a standard ECG, and to estimate stress using the entire ECG waveform rather than just the RR intervals. We also describe methods for estimating sympathetic tone from parasympathetic estimates and observed heart rate using simple and complicated functions. We also describe methods for quantifying the stress level from the parasympathetic and sympathetic indices.

FIG. 1A shows an illustration of an instantaneous lung volume to heart rate (ILV→HR) transfer function, and points out that the area under the positive peak corresponds to parasympathetic responsiveness, while the area above the negative peak corresponds to sympathetic responsiveness.

FIG. 1B shows the entire closed-loop model of circulatory control. The transfer functions instantaneous lung volume to heart rate (ILV→HR), instantaneous lung volume to arterial blood pressure (ILV→ABP) and heart rate (HR) baroreflex can be solved for using cardiovascular system identification (CSI). The line pointing from FIG. 1A to FIG. 1B simply illustrates the idea that only the ILV>HR transfer function is necessary for estimating autonomic nervous system activity.

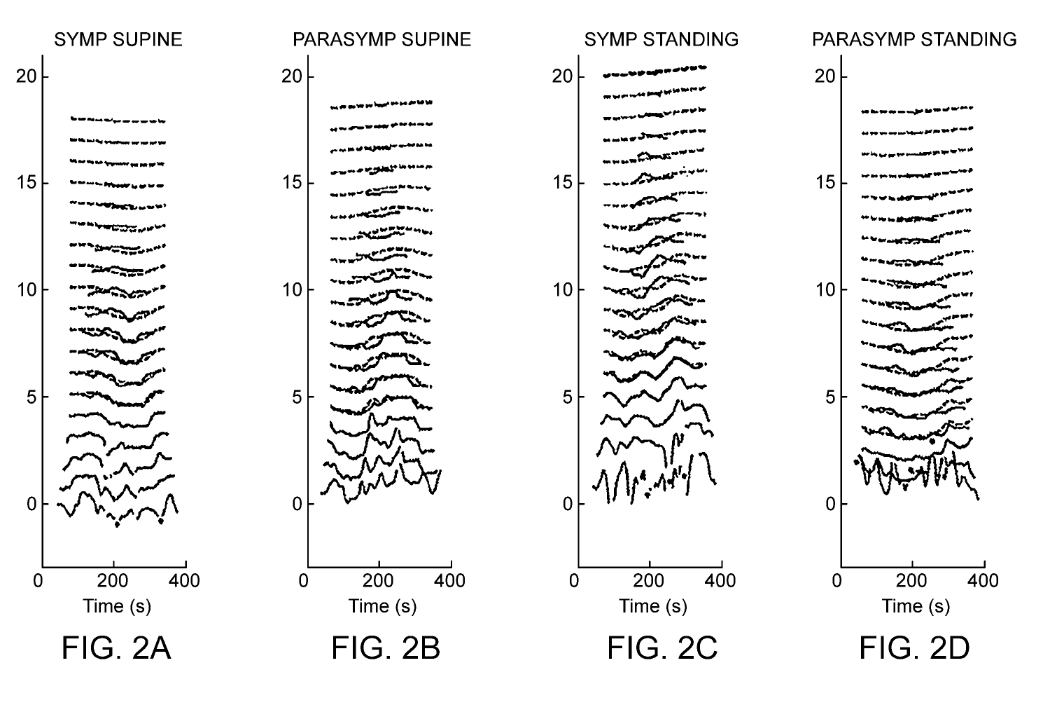

FIG. 2 shows the estimate of sympathetic and parasympathetic tone for supine and standing patients. Weighted principal component regression cardiovascular system identification (WPCR CSI) was run on data windows of increasing length. The bottommost solid lines show estimates where each point on the line was obtained by using the shortest windows. Window length increases from bottom to top, so the top lines show the estimates using the longest windows. The dashed lines are obtained by taking the first reliable estimate obtained with short windows and running a smoothing operation on it. This serves to show that the effect of using more data when running the CSI program is similar to that of averaging estimates obtained using shorter, overlapping windows. This supports the validity of using WPCR CSI on short timescales. The shortest windows to produce reliable estimates were on the order of 2 minutes. Panels A, B, C, and D show the results for sympathetic and parasympathetic in the supine position, and sympathetic and parasympathetic in the standing position, respectively.

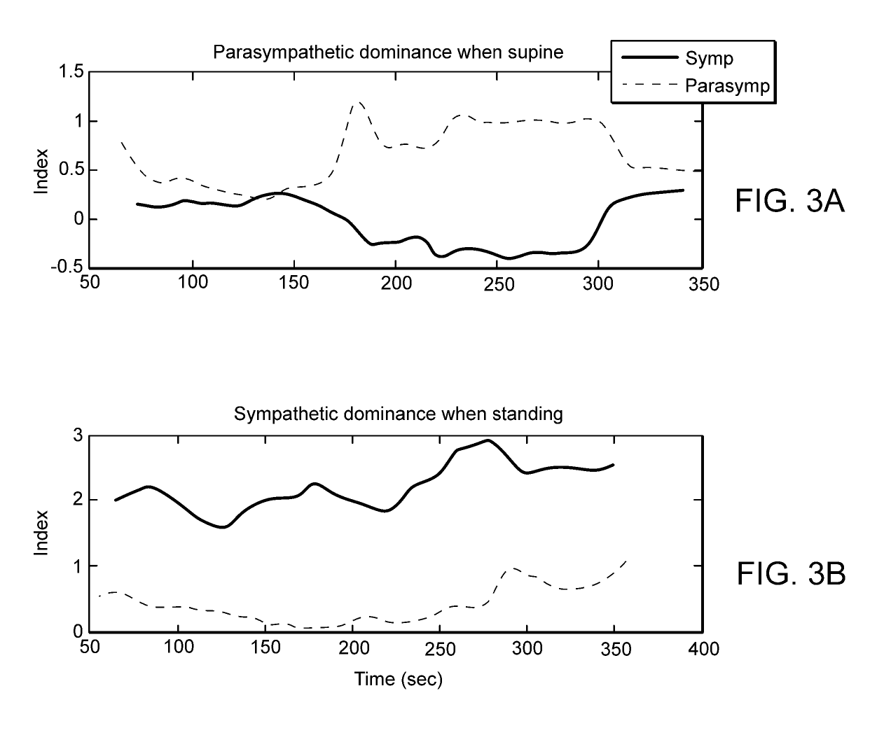

FIG. 3 shows the short-window estimate of parasympathetic and sympathetic function for a supine subject in A and for a standing subject in B. In panel A, we see the expected result, the autonomic tone is fairly constant with respect to time and parasympathetic tone dominates over sympathetic (consistent with the person being at rest). In panel B, we also see the expected result, except that for the standing condition, sympathetic tone dominates over parasympathetic. This implies that standing is a more stressful state than lying down, which is consistent with intuition and physiology.

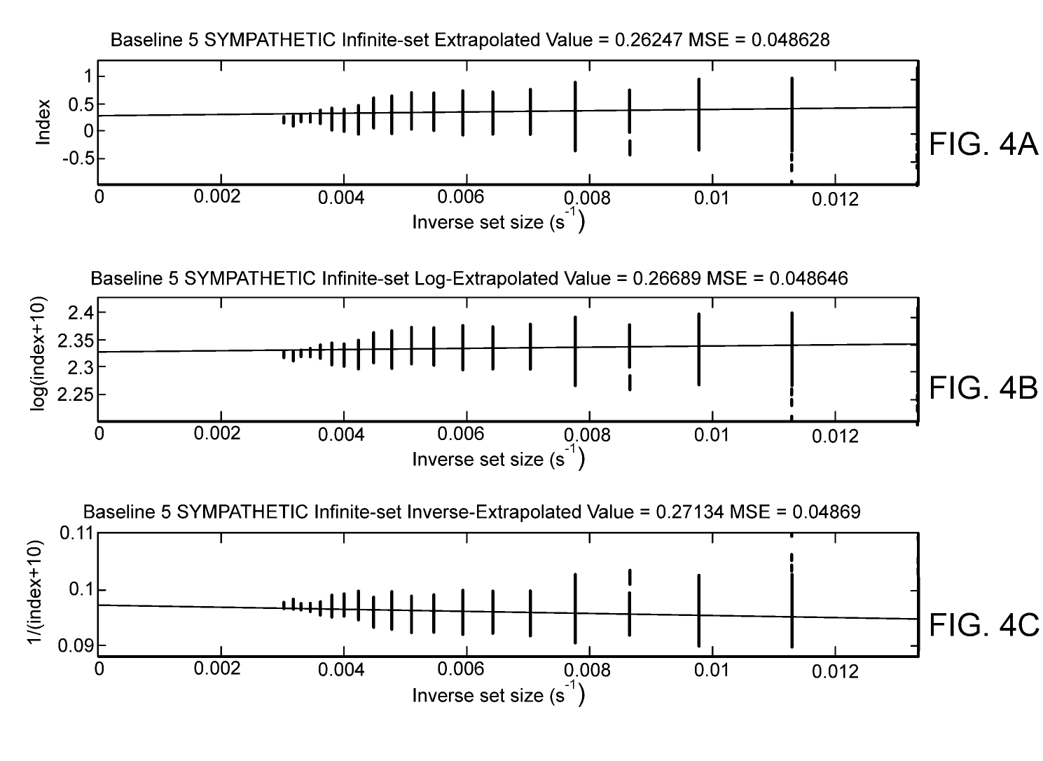

FIG. 4A shows an extrapolation procedure that can be used with CSI to improve the performance on limited amounts of data. The x axis corresponds to the inverse of the data window size, so longer windows are to the left and shorter windows are to the right. For each window size, there are multiple points. Each point corresponds to an estimate of sympathetic tone for overlapping windows over the entire data record. A straight line has been fitted to the data and extrapolated to find the y-intercept. This y-intercept represents an estimate of sympathetic tone for this data record if an infinitely long window of data were available. The title of the panel shows the infinite data window extrapolated value and the mean squared error of the fitted line to the data.

FIG. 4B is similar to 4A except that the natural logarithm of the y-values in 4A is plotted.

FIG. 4C is similar to 4A except that the inverse of the y-values in 4A is plotted.

All three panels show that a similar infinite data set extrapolated value is obtained. The method with the lowest mean squared error was chosen when representing results.

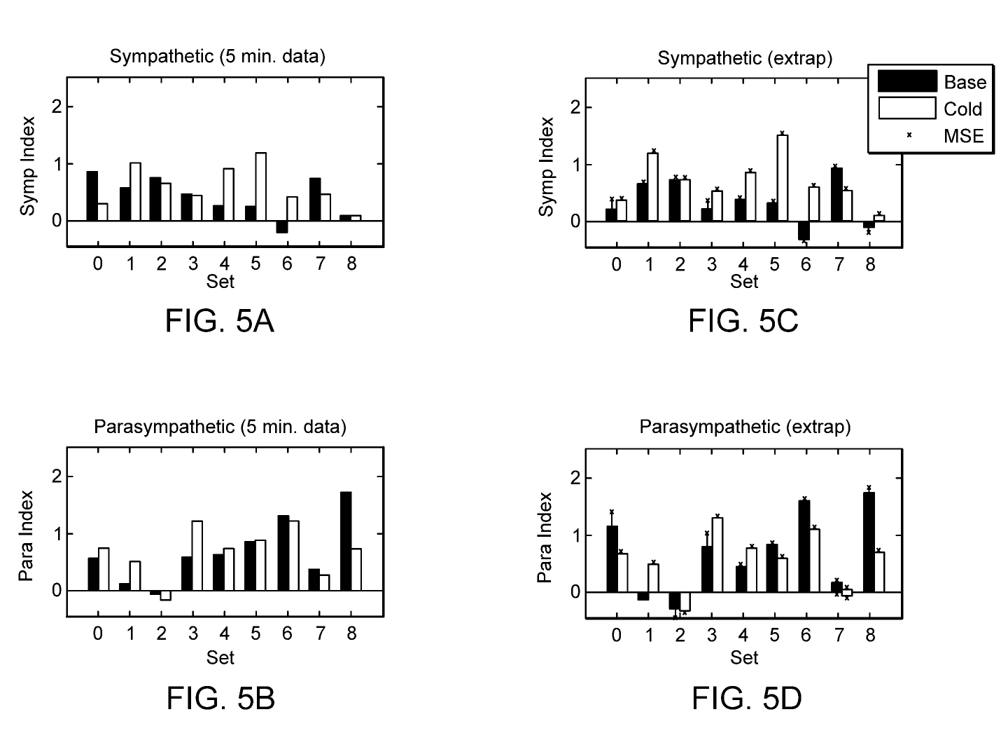

FIG. 5A shows the value of the sympathetic estimate for each of 8 subjects at rest (baseline) and during a cold pressor test (model of stress). The sympathetic values were obtained by running WPCR CSI on the entire data set, comprising about 5 minutes of electrocardiogram and instantaneous lung volume data.

FIG. 5B is similar to 5A, but the parasympathetic estimate is shown. It was calculated in the same way as the sympathetic.

FIG. 5C shows the infinite data set extrapolated values for sympathetic activation. The extrapolation was done as illustrated in FIG. 4. After the extrapolation step, this data showed a statistically significant difference between the baseline and cold pressor conditions, with the cold pressor corresponding to stress (higher sympathetic).

FIG. 5D is similar to 5C, except that it shows the infinite data set extrapolated values of parasympathetic activation. Again, the extrapolation made these results show a statistically significant difference between baseline and cold pressor, with the cold pressor corresponding to stress (lower parasympathetic).

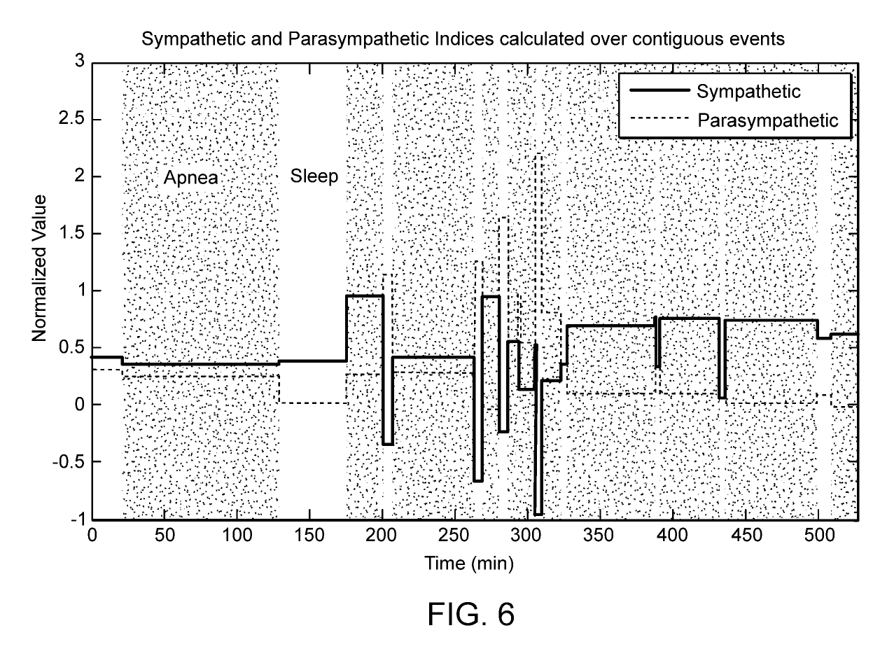

FIG. 6 shows the result of estimating sympathetic and parasympathetic tone via WPCR CSI on data from a patient suffering from sleep apnea. The data set was professionally annotated, and segments of sleep are shown with a white background in the plot while segments of apnea are shown with a gray background. WPCR CSI was computed on each segment separately, and the values are shown with solid and dashed lines. In this particular patient, we observed the expected results: high parasympathetic and low sympathetic activity during sleep, and high sympathetic and low parasympathetic activity during apnea. This implies that apnea may be a good model for stress; however, upon studying seven other patients with sleep apnea, the results were not as clean as the ones shown in this figure, most probably owing to the many varieties and severities of this medical condition.

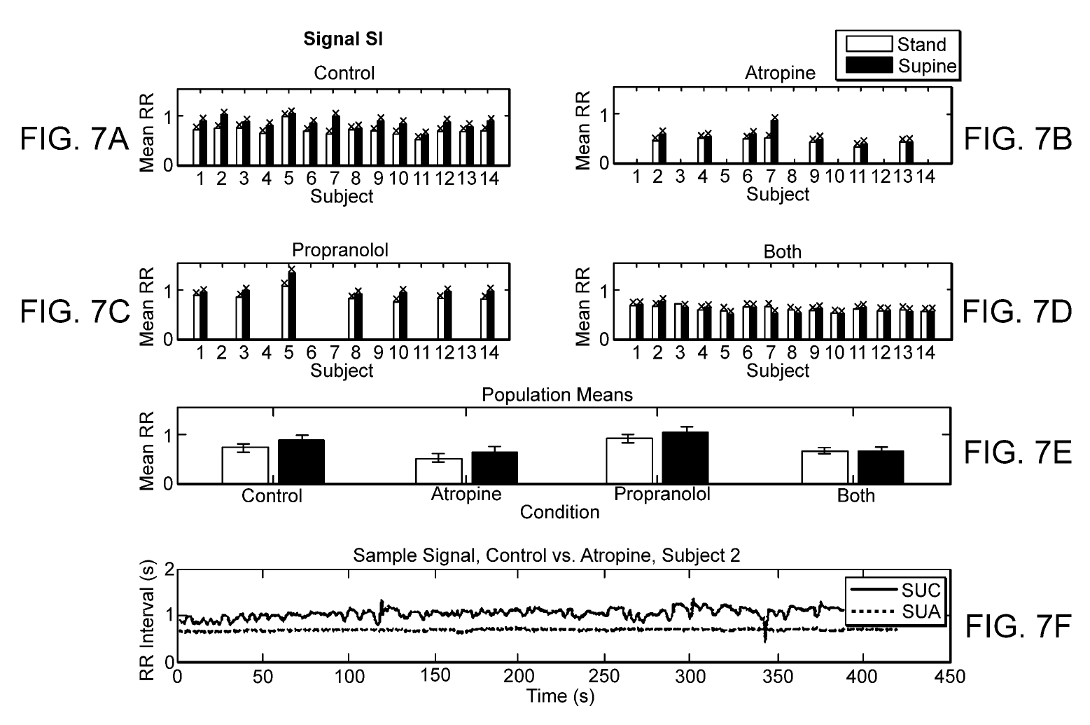

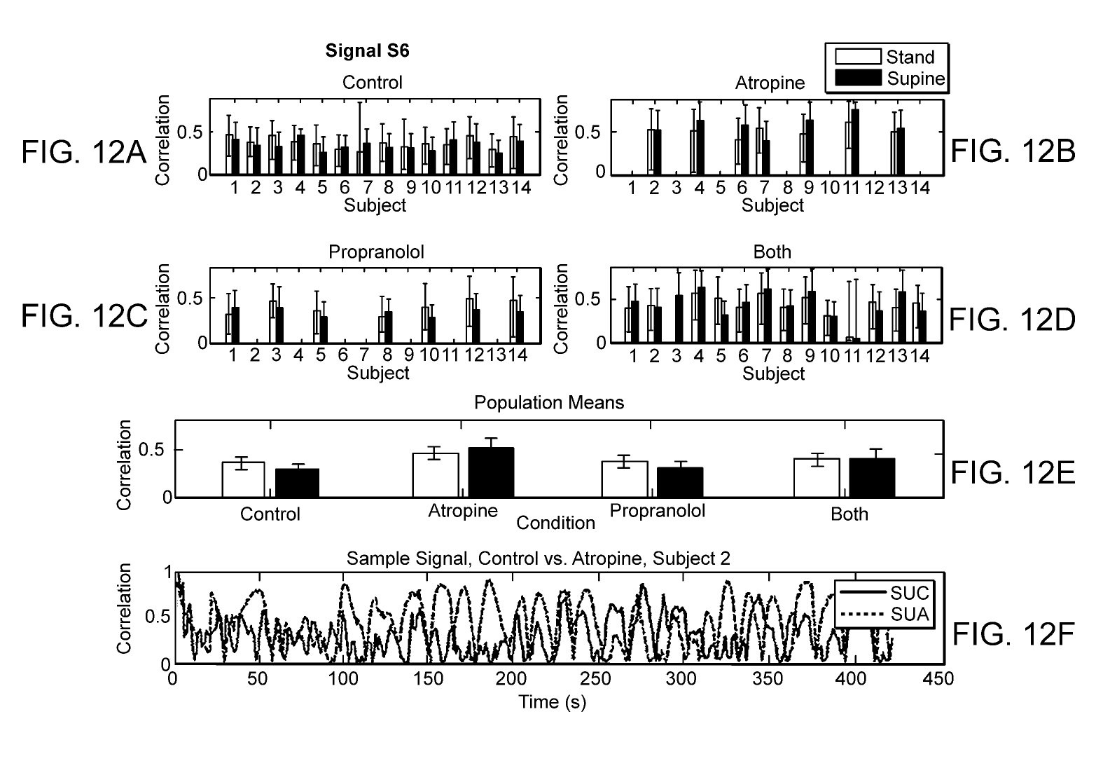

FIGS. 7-12 all have the same format. They show the population results for the time-domain signals S1-S6. In panels A-D, each bar represents the mean value of the signal over the course of the entire data record, with standard deviation being shown by the error bars. Each set of two bars corresponds to a particular subject, numbered from 1 to 14. The estimates for the standing data are shown in gray and the estimates for supine data are shown in black. Panel A is the control condition, in which no pharmacologic intervention was used. Panel B shows the results under atropine, which blocks parasympathetic activity. Panel C shows the results under propranolol, which blocks sympathetic activity, and panel D shows the results under double blockade, in which propranolol and atropine were administered together. Panel E shows the summary of panels A-C. The height of each bar represents the mean and the error bars show the standard deviation of the means across subjects. This allows us to see if signal properties are well conserved among subjects in general. Panel F shows a sample representation of the particular signal as a function of time for one subject. The solid line shows the supine control (where we expect high parasympathetic activity) and the dashed line shows supine atropine (where we expect low parasympathetic activity, since it is inhibited by atropine). Since most of the methods are sensitive to parasympathetic activity, we expect the control and propranolol conditions to be about the same, while the atropine and double blockade conditions are expected to be similar to each other, but lower than the control and propranolol. This is observed in all figures except 12, since FIG. 12 shows the results of S6, which seems more correlated with sympathetic activity rather than parasympathetic. In this case, we expect control and atropine to be similar, and propranolol and double blockade to be similar to each other and lower than control.

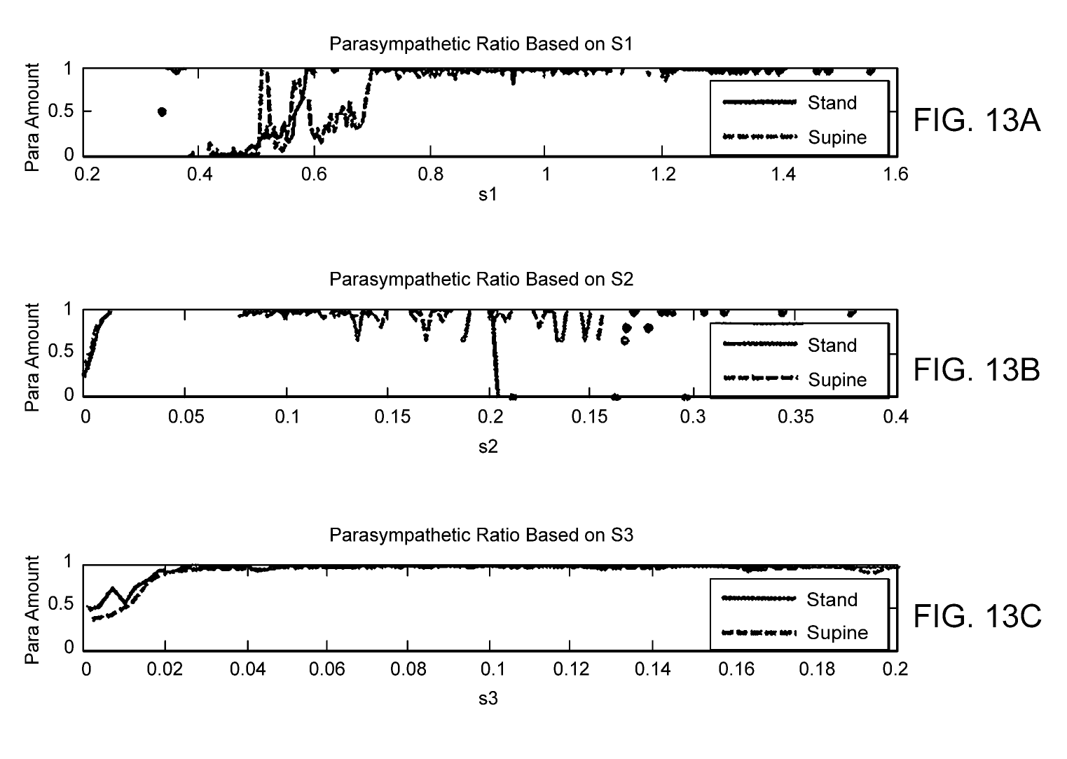

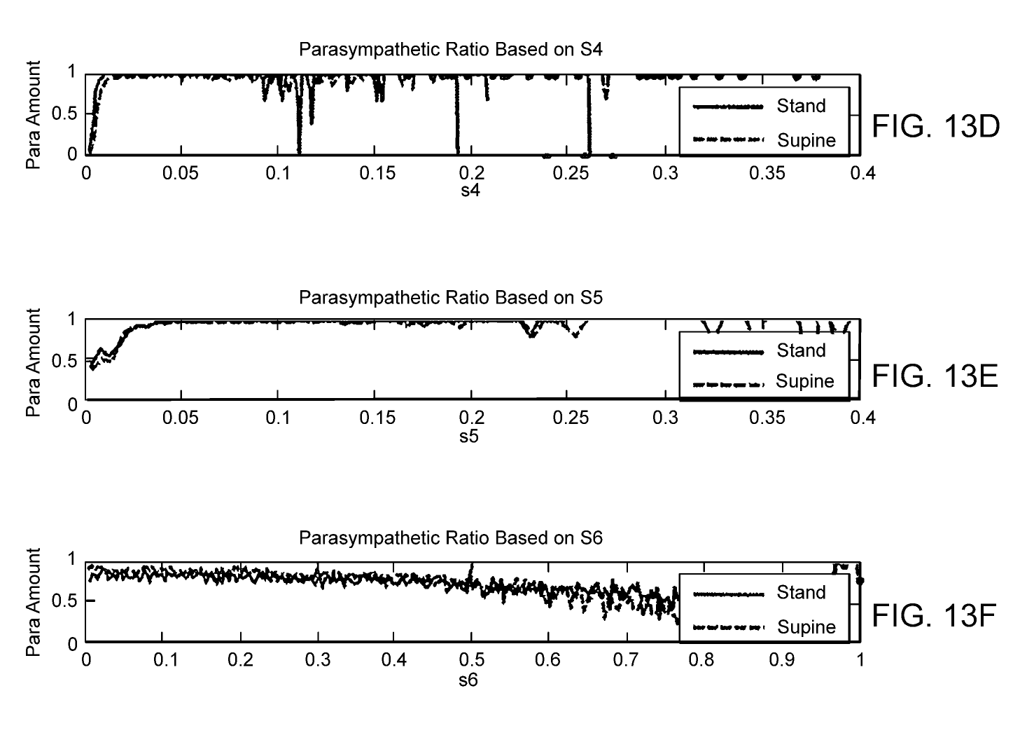

FIG. 13A shows the parasympathetic ratio for S1 in the standing (solid line) and supine (dashed line) conditions. Parasympathetic ratio was defined as the signal value in a parasympathetic only state divided by the sum of the signal value in the parasympathetic only state plus the signal value in the sympathetic only state. The purpose of this function is to provide a normalized estimate of parasympathetic activity based on the raw signal value. From FIG. 13A, we see that a value of 0.2 for S1 corresponds to no parasympathetic activity, whereas a value of 0.8 corresponds to complete parasympathetic activity.

FIG. 13B is the same as 13A, but for S2.

FIG. 13C is the same as 13A, but for S3.

FIG. 13D is the same as 13A, but for S4.

FIG. 13E is the same as 13A, but for S5.

FIG. 13F is the same as 13A, but for S6.

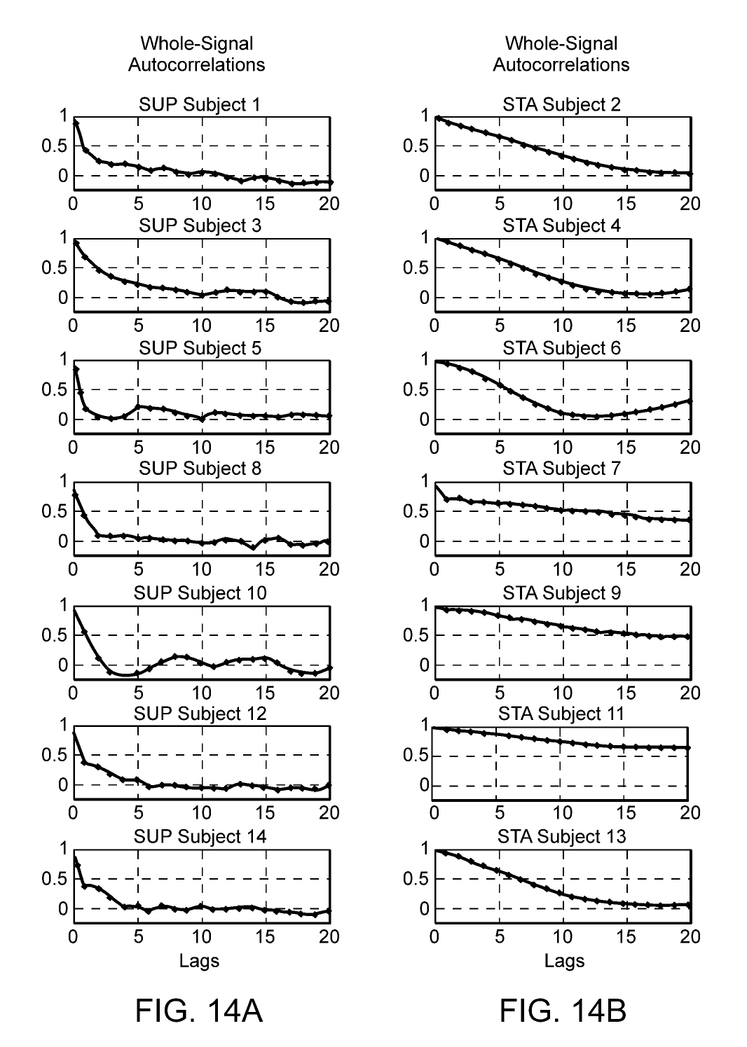

FIG. 14 shows the whole signal autocorrelations for supine propranolol (blocked sympathetic) subjects on the left in A and standing atropine (blocked parasympathetic) subjects on the right in B. For all the subjects in panel A, we observe much more pointed autocorrelations than for the subjects in B. This matches theory since parasympathetic activity is more uncorrelated, so it should yield peaked autocorrelations when present, and more smooth autocorrelations when absent (as in B). The autocorrelations in this figure were computed using approximately 5 minutes of data.

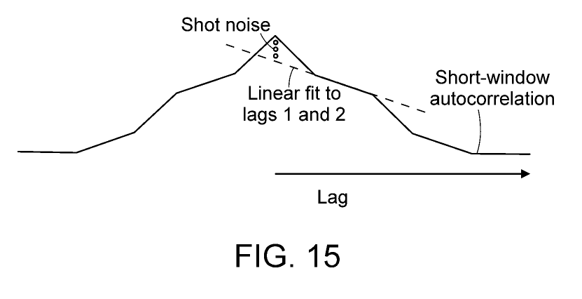

FIG. 15 shows how shot noise is calculated. The solid peaked line shows a theoretical autocorrelation function. The dashed line shows the result of a linear fit to the autocorrelation value for lags 1 and 2. Shot noise is the difference between the peak of the autocorrelation at lag 0 and the extrapolated linear fit to lags 1 and 2.

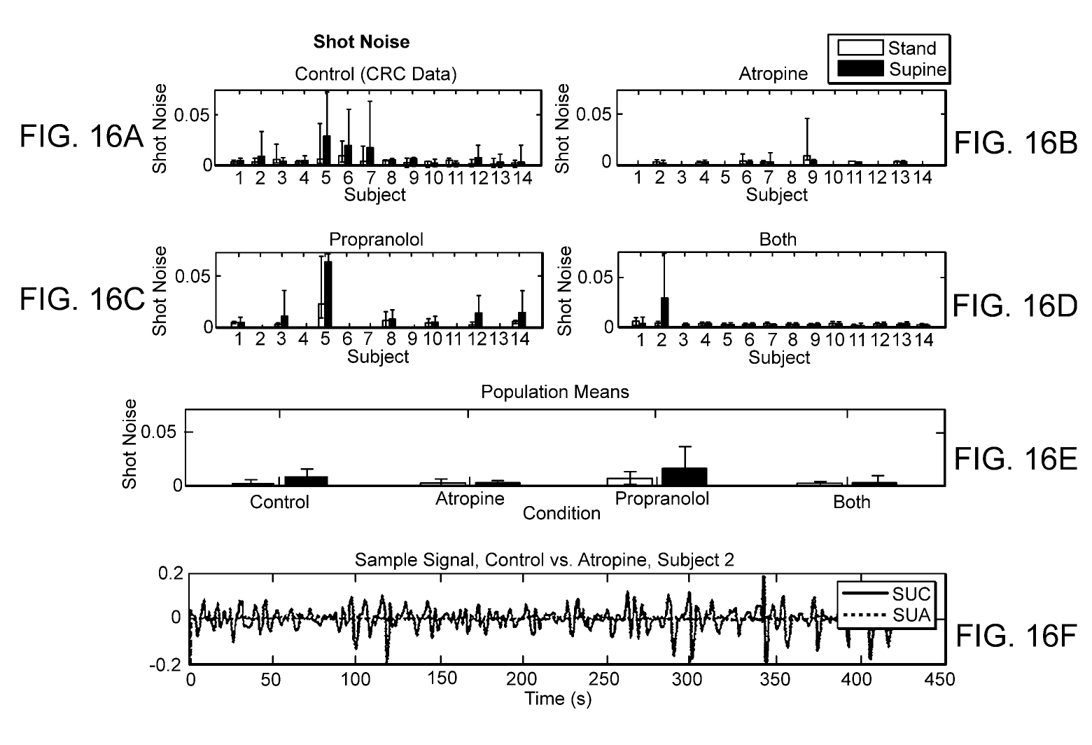

FIG. 16 has the same data as FIGS. 7-12, except it is for shot noise. Here we see that the linear fit to lags 1 and 2 may not be the best way to approximate shot noise since we get negative values. This implies that the autocorrelation has high values for lags 0 and 1, and a low value for lag 2, causing the extrapolation to overshoot the autocorrelation peak. More complicated estimates of shot noise, as discussed in the preferred embodiment, would correct this problem, or it could be circumvented by simply taking the absolute value of the linearly approximated shot noise. Since shot noise estimates parasympathetic activity, we expect high values for control and propranolol, and low values for atropine and double blockade. This is manifested as high standard deviations in panels A and C and low standard deviations in panels B and D (since taking the absolute value of a high-standard deviation signal will give a high mean).

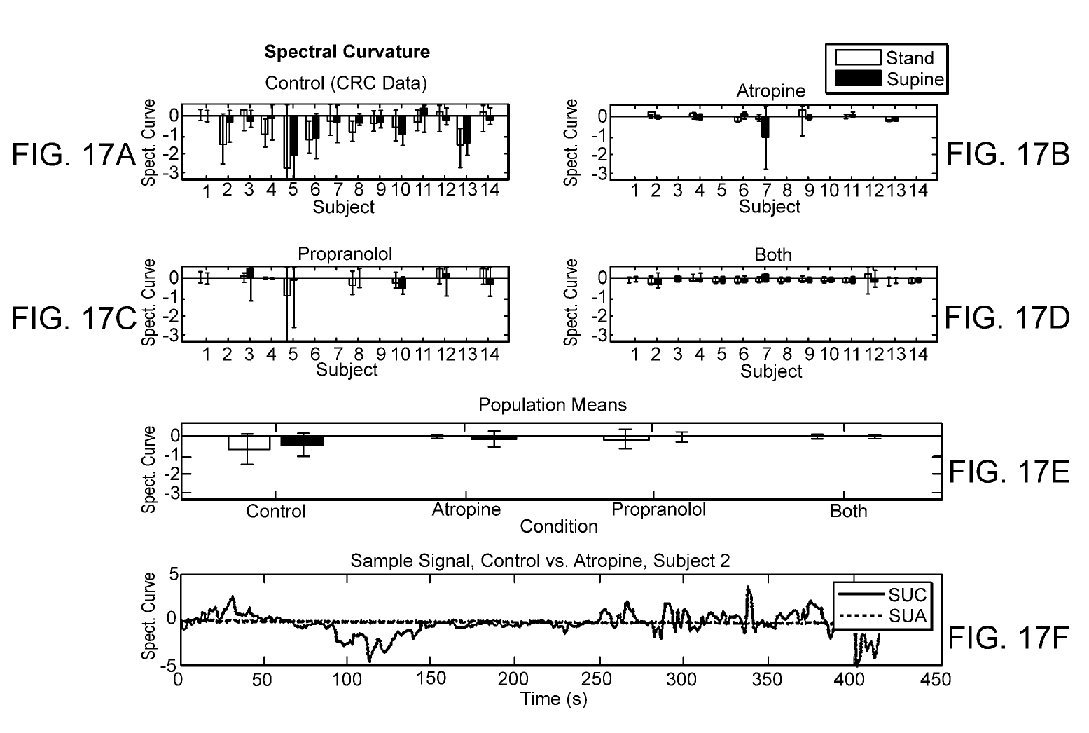

FIG. 17 shows the same data as FIGS. 7-12 and 16, but for the spectral curvature results. Spectral curvature gives primarily negative values with large standard deviations in the control case, and zero values under atropine and double blockade. The propranolol data shows some negative and some positive values, which cancel out when computing the population mean (E). This implies that spectral curvature is sensitive to parasympathetic activation, and when parasympathetic activity is blocked, spectral curvature goes to zero.

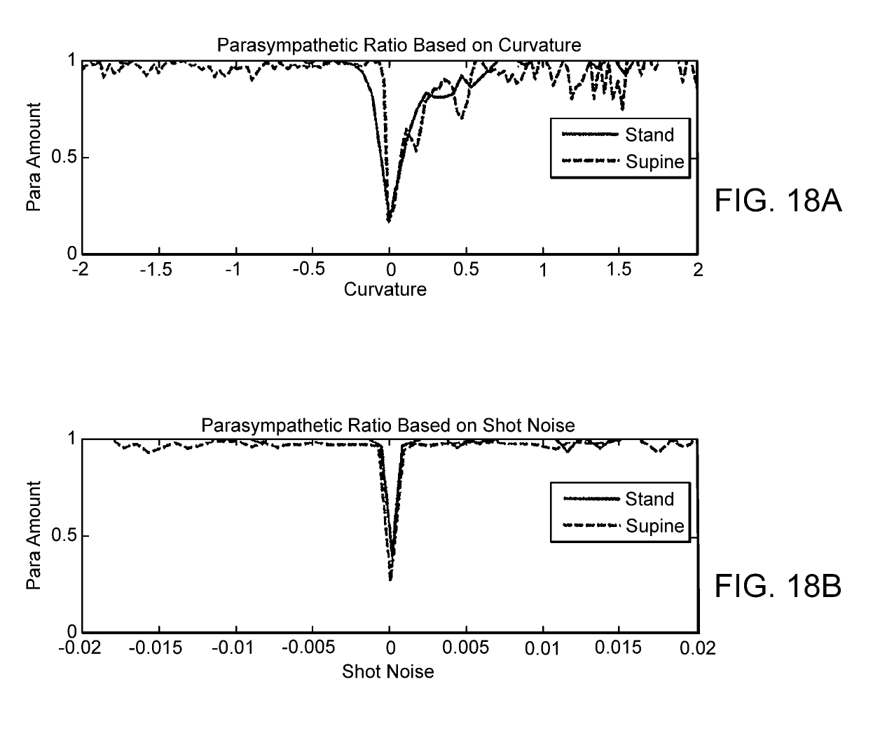

FIG. 18A shows the parasympathetic ratio based on spectral curvature. This figure shows that spectral curvature values close to 0 represent low parasympathetic activity and more positive and negative values correspond to higher parasympathetic activity. FIG. 18B shows the parasympathetic ratio for shot noise. Again, we see that large absolute values of shot noise correspond to high parasympathetic activity, whereas values of shot noise close to 0 correspond to low parasympathetic activity.

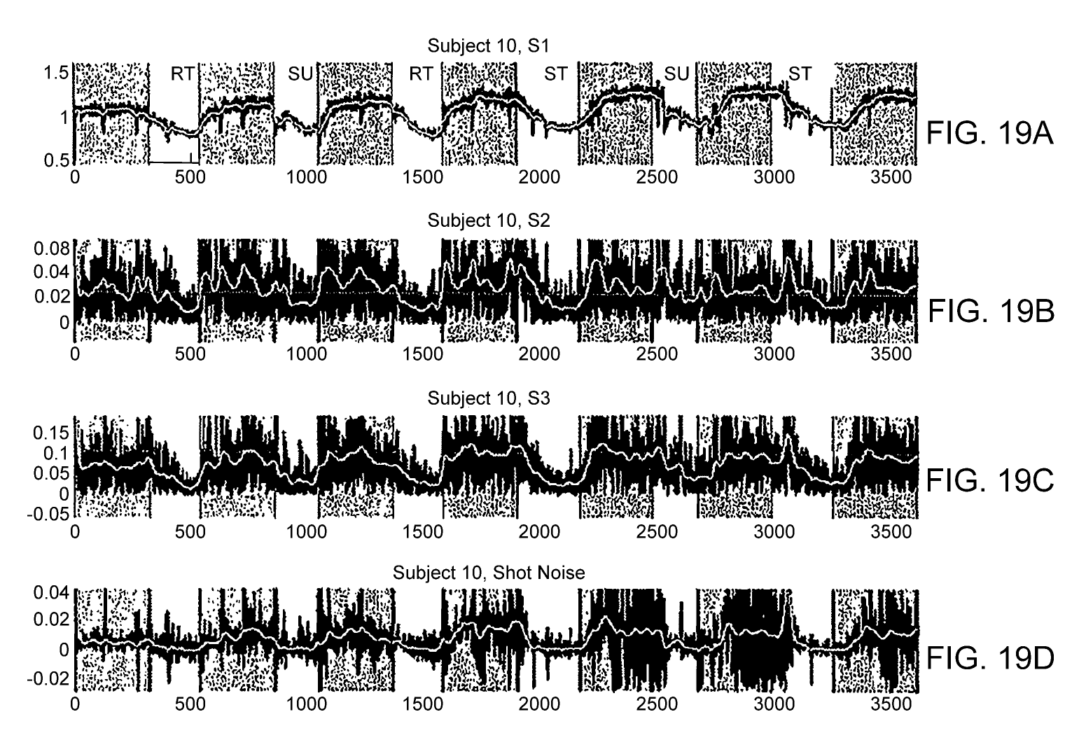

FIG. 19 shows the results of S1, S2, S3, and Shot Noise in panels A, B, C and D respectively. The data in this case was a continuous recording of a patient undergoing a dynamic tilt-table experiment. All the gray regions of time correspond to the supine position, the regions labeled ST in panel A correspond to a slow tilt up, the regions labeled RT correspond to a rapid tilt up, and the regions labeled as SU correspond to the patient standing up. We expect to see higher sympathetic activity during the up-tilts and standing than during the supine portions. This can be seen in the raw signals (black trace) and their smoothed version (gray trace). A distinct difference can be observed in the values of the signals during the supine and intervention portions of time.

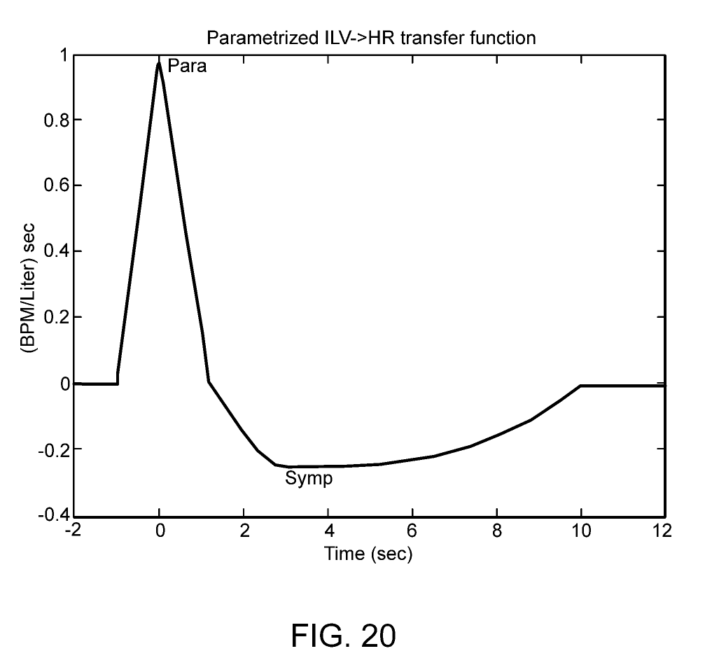

FIG. 20 shows the parameterized ILV→HR transfer function. The transfer function is fully specified by the parasympathetic value that defines the positive peak, and the sympathetic value that defines the negative peak. In this particular form of the function, the general shape is fixed in a particular way, but as specified in the preferred embodiment, any alterations that preserve the qualitative shape of the function are covered in the scope of this invention.

In one preferred embodiment this invention relates to the method of estimating physiological stress in a subject by analysis of intervals between heart beats. The signals considered are the actual interval, the instantaneous slope in heart rate, the second difference of heart rate, the mean of the absolute first difference within a window, the normalized interval, the correlation coefficient of an interval series within a window, a shot-noise estimate using short autocorrelations, the spectral curvature of an extended autocorrelation, cardiovascular system identification, hidden Markov model, integrate and fire model, and complex electrochemical model. These signals can be combined to reduce noise, and can be used to estimate the amount of stress.

The method of the invention, in one aspect, includes detecting a physiologic signal in a subject and processing this signal to obtain a sequence of intervals corresponding to time intervals between heart beats. The relation of these inter-beat intervals is evaluated in real time or in longer time-windows to provide an estimate of the subject's stress level. In a preferred embodiment, the physiologic signal is a real-time electrocardiogram and the time-windows are on the order of one to ten seconds. Regardless of the source of the physiologic signal, these embodiments include using the actual inter-beat intervals, referred to as signal 1 or S1, as an indication of parasympathetic tone, with larger intervals corresponding to higher parasympathetic activation. S1 results are shown in FIG. 7. In general, S1 shows larger values when parasympathetic activity is high (control and propranolol) and shows smaller values when parasympathetic activity is inhibited (atropine and double blockade).

In yet another embodiment, the instantaneous slope in heart rate, which will be referred to as S2, can be calculated according to the equation

$$S {2}_{k} = \vert \frac{( {HR}_{k} - {HR}_{k - 1} ) + ( {HR}_{k + 1} - {HR}_{k} )}{2 ( ( {HR}_{k - 1} + {HR}_{k + 1} ) / 2 )} \vert = \vert \frac{{HR}_{k + 1} - {HR}_{k - 1}}{{HR}_{k - 1} + {HR}_{k + 1}} \vert \qquad Eq . 1$$

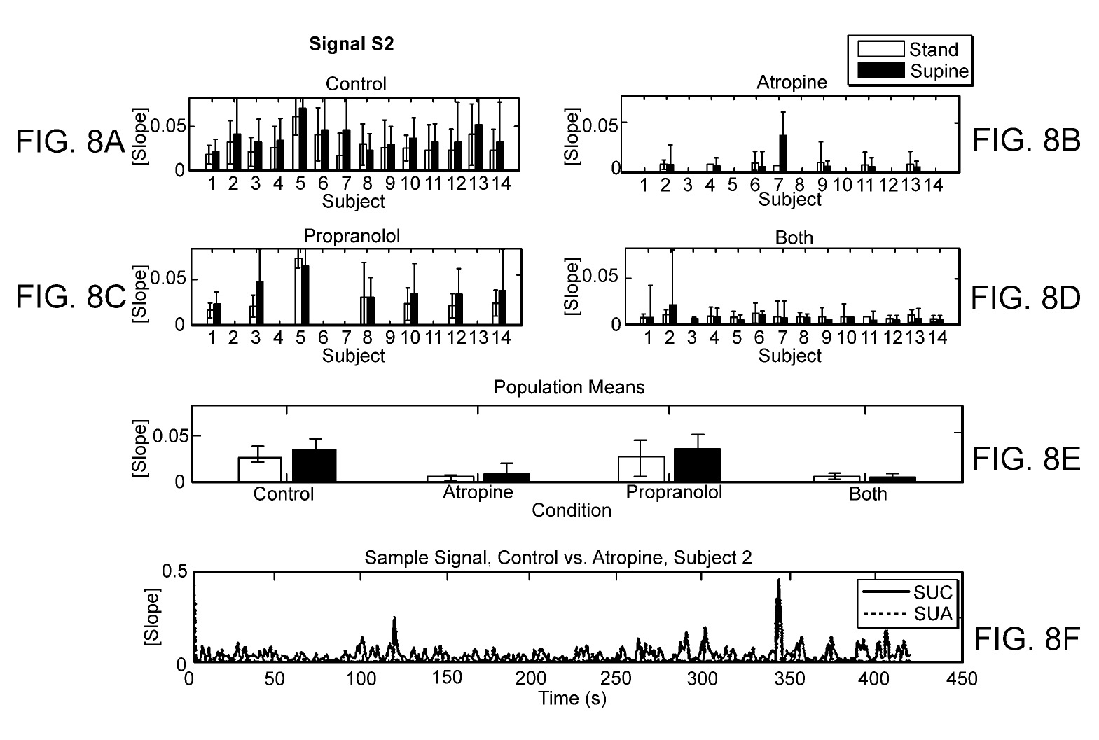

In this equation, HRₖ is the instantaneous heart rate in beats per minute, calculated as 60/RRₖ, where RRₖ is the k^{th} inter-beat interval measured in seconds, k is an index corresponding to the chronological order of the inter-beat intervals, and S2ₖ is the value of signal 2 at index k. In another embodiment, the normalizing denominator of the rightmost equation may be absent or replaced by a constant. In yet another embodiment, the raw inter-beat intervals may be used instead of the instantaneous heart rate. It has been shown that S2 is directly proportional to the strength of parasympathetic activity, as seen in FIG. 8.

In yet another embodiment, the second difference of the inter beat intervals, referred to as S3, can be calculated according to the equation

$$S {3}_{k} = \vert \frac{( {HR}_{k + 1} - {HR}_{k} ) - ( {HR}_{k} - {HR}_{k - 1} )}{( {HR}_{k - 1} + {HR}_{k} + {HR}_{k + 1} ) / 3} \vert = \vert \frac{- 2 {HR}_{k} + {HR}_{k - 1} + {HR}_{k + 1}}{( {HR}_{k - 1} + {HR}_{k} + {HR}_{k + 1} ) / 3} \vert \qquad Eq . 2$$

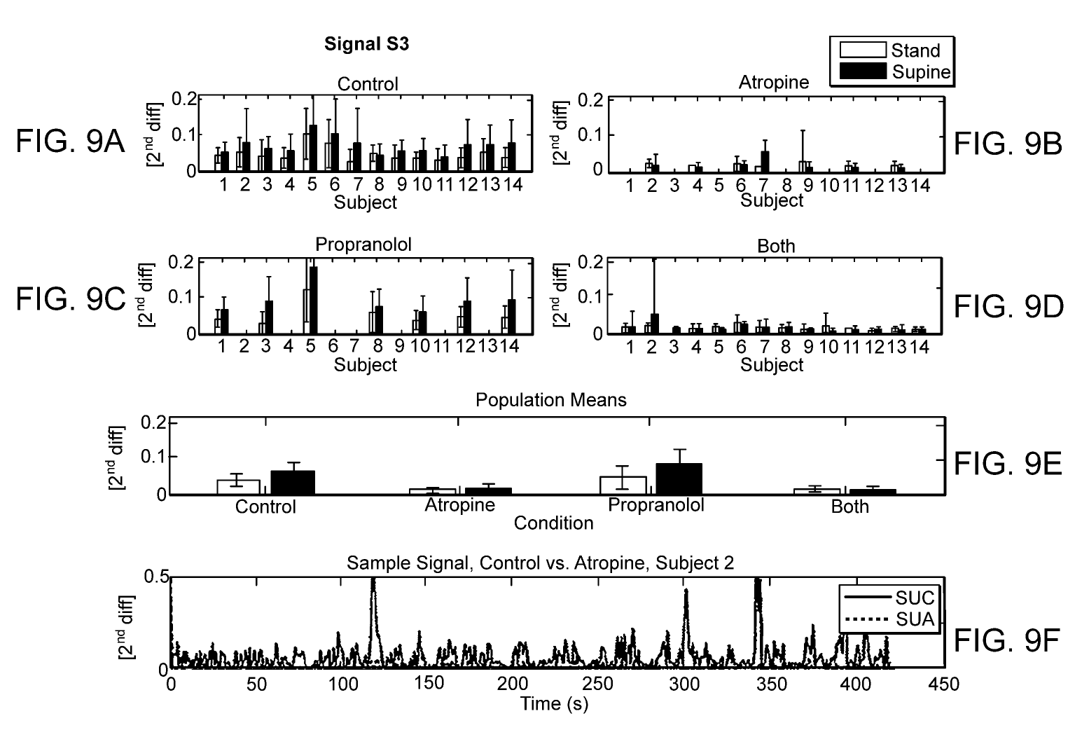

In this equation, HRₖ and k have the same meaning as defined above for S2, and S3ₖ is the value of S3 at the time corresponding to index k. In another embodiment, the normalizing denominator may be absent or replaced by a constant. In yet another embodiment, the raw inter-beat intervals may be used instead of the instantaneous heart rate. In yet another embodiment, a particular value of S3 may be calculated using more than 3 consecutive HR values. S3 has been shown as directly proportional to the magnitude of parasympathetic activity, as seen in FIG. 9.

In yet another embodiment, the mean absolute first difference, referred to as S4, can be obtained according to the equation

$$S {4}_{k} = {\sum }_{i = k - ⌊ n / 2 ⌋}^{k + ⌊ n / 2 ⌋ - 1} \frac{\vert {RR}_{i + 1} - {RR}_{i} \vert}{n - 1} \qquad Eq . 3$$

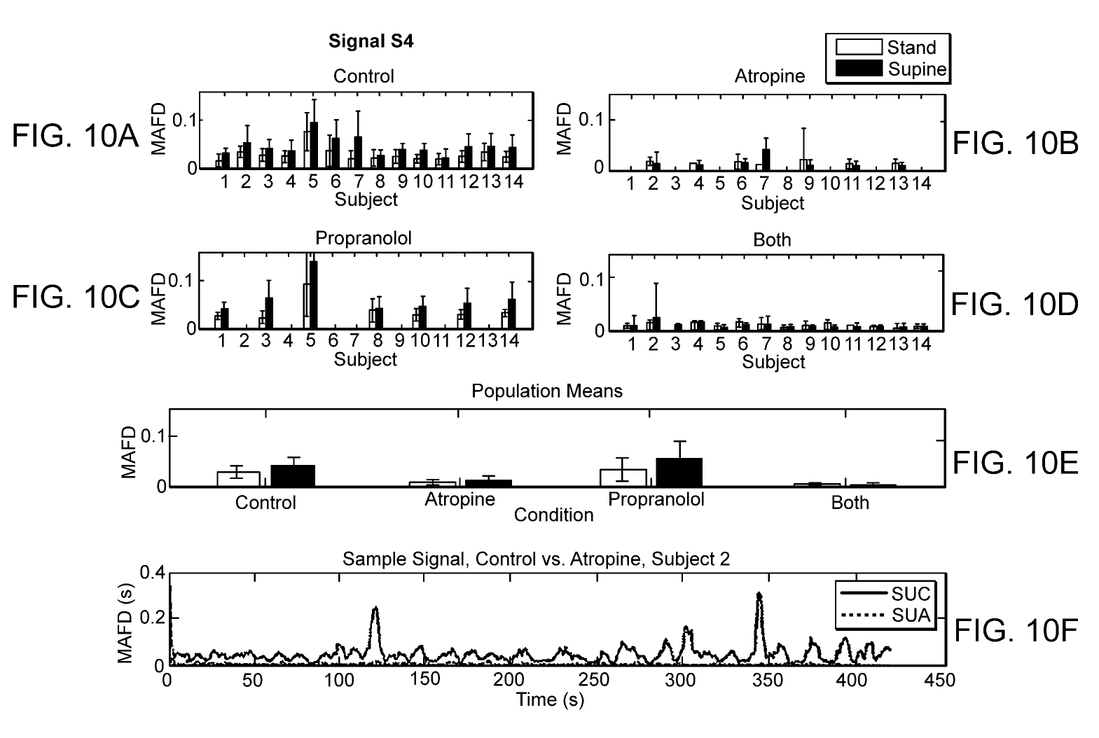

In this equation, n is the size of the relevant window, on the order of 5 to 10 inter-beat intervals, and RR and k are defined as above. S4ₖ is the value of S4 at the time corresponding to the k^{th} time index. In another embodiment RR may be replaced with HR. S4 has been shown to be directly proportional to parasympathetic tone, as shown in FIG. 10.

In yet another embodiment, the inter-beat interval differences can be normalized such that they represent a percent change per unit time. This signal is referred to as S5, and is calculated according to the equation

$$S {5}_{k} = ( \frac{{RR}_{k + 1} - {RR}_{k}}{{RR}_{k}} ) / {RR}_{k + 1} \qquad Eq . 4$$

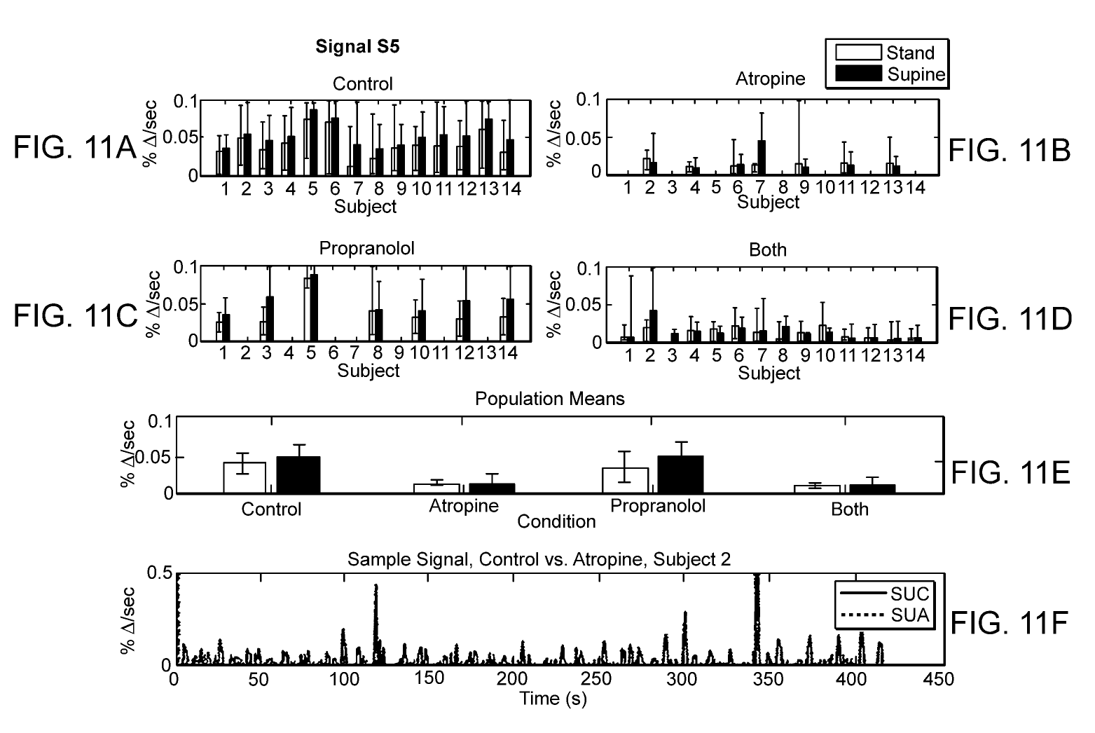

In this equation, RR and k are defined as above, and S5ₖ is the value of S5 at the time corresponding to the k^{th} time index. In another embodiment, HR may be substituted for RR. In yet another embodiment, the normalizing denominator RRₖ₊₁ may be absent or replaced by a constant. S5 has been observed to directly correlate to the strength of parasympathetic activation, as illustrated in FIG. 11.

In yet another embodiment, the linear correlation coefficient of a sequence of inter-beat intervals, referred to as S6, can be calculated according to

$$S {6}_{k} = \vert \frac{cov ( \overline{X} , {\overline{R}}_{k} )}{{σ}_{\overline{X}} {σ}_{{\overline{R}}_{k}}} \vert ; \overline{X} = [ 1 , 2 , 3 , … N ] , {\overline{R}}_{k} = [ {RR}_{k - N / 2} , {RR}_{k - N / 2 + 1} , … {RR}_{k + N / 2} ] \qquad Eq . 5$$

In this equation, cov(.) is the covariance of the two arguments and σ is the standard deviation of the subscripted argument. The X and R vectors are defined on the right of the equation. N is the number of RR intervals that fit within a specified time window, on the order of 20 seconds. In yet another embodiment, the linear correlation coefficient may be replaced by the mean squared error around a higher-order fit of the same two sequences. In another embodiment, the goodness-of-fit may be calculated in another way (for example absolute error). S6 has been shown to weakly correlate to the strength of sympathetic activity, as seen by the data in FIG. 12.

In yet another embodiment, the degree of randomness, termed shot noise, can be calculated from the inter-beat interval series in short windows containing about 5 RR intervals. Shot noise is calculated as the difference between the O-lag value of the autocorrelation of the short RR sequence and the linear extrapolation to lag 0 of the lag 1 and lag 2 values of the autocorrelation, as illustrated in FIG. 15. A high absolute value and variance of shot noise are observed to correlate to high parasympathetic activity, as shown in FIG. 16.

In yet another embodiment, an unconventional autocorrelation function can be calculated from the inter-beat interval sequence. To compute this autocorrelation, a short window of RR intervals is convolved with a time-reversed longer window of RR intervals, both windows being taken from the entire RR interval sequence such that the middle value in each window corresponds to the same sample in the RR interval sequence. The shorter window can contain on the order of 5 intervals and the longer window can contain on the order of 30 intervals. In another embodiment, the frequency spectrum of this autocorrelation function can be calculated in any way known to practitioners of the art, such as, but not limited to, the fast Fourier transform. In yet another embodiment, the average spectral energy can be calculated in three frequency bins. The three frequency bins may contain frequency components on the approximate ranges 0 to 0.04 Hz, 0.04 to 0.125 Hz, and 0.125 to 0.4 Hz. In yet another embodiment, the second difference of the three average energy values in the said frequency bins can be calculated to yield a single value representing the spectral curvature of the RR sequence corresponding to the time on which the short and long windows were centered. The spectral curvature has been observed to have large negative values when parasympathetic and sympathetic activity are normal, and to have values close to zero when either sympathetic or parasympathetic branches dominate, or if both are eliminated, as shown in FIG. 17. This method may be a useful indicator of the presence of an autonomic system imbalance, caused by pharmacologic intervention or altered stress state.

In another embodiment, the raw value of S1-S6 is transformed to a value of parasympathetic activation by means of a parasympathetic ratio. This ratio can be computed by dividing the value of a particular signal in a parasympathetically dominated intervention (such as supine propranolol) by the sum of that same value plus the value of that same signal in a sympathetically dominated intervention (such as standing atropine). This ratio would give a value of 1 if a particular signal matches a pure parasympathetic state, a value of 0 if the signal matches a purely sympathetic state, and an intermediate value for combinations between these two extremes. Calculated parasympathetic ratio functions for S1-S6 are shown in FIG. 13 A-F and for spectral curvature and shot noise are shown in FIG. 18 A-B. These figures show the parasympathetic ratios computed in the standing and supine states separately (the ratio was propranolol divided by propranolol plus atropine). The function that was eventually used was the average of the two lines. In another embodiment, a different mapping function may be used to convert from the raw signal value to a more meaningful, normalized autonomic nervous system function value.

In another embodiment, an additional physiologic signal may be recorded, or the said signal may be derived from the electrocardiogram as described in the literature. The said additional signal is the subject's instantaneous lung volume, synchronized in time to the electrocardiogram. In another embodiment, weighted principal component regression cardiovascular system identification (WPCR CSI), as described by Xinshu Xiao, can be used to calculate the parasympathetic and sympathetic indices from the transfer function relating instantaneous lung volume to heart rate [7]. In one embodiment, the WPCR CSI method can be run on an entire data set of approximately five minutes or longer to obtain a steady-state estimate of autonomic system function. In another embodiment, the said method could be run on contiguous segments of data regardless of segment length. For example, WPCR CSI was run on entire portions of sleep apnea data, with each sleep segment and each apnea segment being treated as a single data trace. As shown in FIG. 6, the parasympathetic values peaked during sleep and sympathetic values peaked during apneic episodes for this particular patient. These results are consistent with a stressed state during apnea, and a relaxed state during sleep.

In yet another embodiment, the said method can be run on shorter (approximately 2 minutes of data), overlapping data segments in order to obtain a time-dependent estimate of autonomic system function. The result of running CSI on short windows of data is shown in FIG. 2. This figure illustrates the idea that the short segments provide a valid time-localized estimate of sympathetic and parasympathetic activity since smoothed short-window estimates match the long-window estimate. FIG. 3 shows the efficacy of estimating sympathetic and parasympathetic tone in supine (A) and standing (B) subjects as a function of time. When the subject is supine, the parasympathetic branch is seen to dominate, and when the subject is standing, the sympathetic branch dominates. In yet another embodiment, the results of the short overlapping data segments can be extrapolated to infinite set size to improve the quality of the measure if the autonomic nervous system function can be assumed constant over the duration of the physiologic signals. The extrapolation to infinite set size can be done by plotting the inverse of the window size on the x axis, and a function of the calculated value of the sympathetic or parasympathetic parameters on the y axis. For each x axis value, one would obtain multiple y axis values, corresponding to different shifts of the analysis window through the entire data record. The functions applied to the y values can be linear, such as but not limited to scaling by a nonzero constant, or nonlinear, such as but not limited to the logarithm, inverse, or exponential. The extrapolation procedure is illustrated in FIG. 4. In the studied data set, statistically significant (95% confidence) differences were obtained between the cold pressor (high sympathetic, low parasympathetic) and baseline (low sympathetic, high parasympathetic) interventions using the extrapolation method. Without extrapolation, the expected observations (namely, higher parasympathetic values for baseline and higher sympathetic values for the cold pressor) were made in three of eight subjects. These results are shown in FIG. 5.

In yet another embodiment, the shot noise can be calculated on an entire data record of RR intervals at least five minutes in duration. This method is useful for estimating parasympathetic tone during a long and unchanging intervention; for example, if the subject is lying in the intensive care unit without moving or responding. As seen in FIG. 14, the autocorrelation functions of parasympathetically-dominated interventions are significantly more peaked than those of sympathetically-dominated interventions, meaning that the shot noise value would be greater under parasympathetic dominance than under sympathetic dominance. In another embodiment, the shot noise may be normalized or otherwise related to the absolute value of the autocorrelation for a set value of lag. Again referring to FIG. 14, we see that parasympathetic dominance causes the autocorrelation function to decay to zero for lags of 5-10 samples, whereas sympathetic dominance yields relatively high autocorrelation values even for lags beyond 15 samples. In another embodiment, the ratio of shot noise versus the autocorrelation value at relatively long lags may be used to estimate the stress level directly; this particular embodiment assumes that the sympathetic to parasympathetic ratio is directly proportional to stress.

In yet another embodiment, the needed signals are the instantaneous heart rate, which is obtainable from the electrocardiogram or the RR interval series, and the instantaneous lung volume, also obtainable from the electrocardiogram or directly, and autonomic nervous system function is estimated assuming a Hidden Markov Model. The said model involves the parameterization of the instantaneous lung volume to heart rate transfer function such that it is fully described by two values: a positive parasympathetic peak, and a negative sympathetic peak, as shown in FIG. 20. In another embodiment, the parameterized transfer function may have a qualitatively similar shape to that shown in FIG. 20 but with different values for the locations in time of the start of the function, the zero crossing, and the peaks. Thus, the hidden states in this model are the two-element vectors describing the actual parasympathetic and sympathetic values at each point in time: Sₙ=[para symp]. The observed values are the instantaneous heart rates at each point in time (may be obtained by inverting the RR interval and multiplying by 60 to get beats per minute). We can relate the hidden state to the observed state by convolving the transfer function defined by the state values with the instantaneous lung volume signal at the corresponding times. This yields the “actual” heart rate. In essence, the maximum a posteriori (MAP) estimate requires that we maximize the probability that the observed heart rate occurs given that the hidden states are known, and that the entire time-vector of hidden states is likely |Ŝ_{MAP}=arg maxₛP(HR|S)P(S). To calculate this quantity, we also define a transition probability, which defines the probability that a particular state vector at time n will change to any other possible state vector at time n+1. For example, it is known that parasympathetic activity can change very rapidly whereas sympathetic activity changes much more slowly with time. Thus, the large changes in the parasympathetic value of the state vector are more likely than large changes in the sympathetic value, and the time-series of states must reflect this in order to have a high probability. The state transition probabilities were defined as symmetric exponential functions around 0, with specific sympathetic and parasympathetic parameters, as shown by the following equations:

$$P ( {S}_{n} \vert {S}_{n - 1} ) = {e}^{\frac{- \sqrt{2} \vert Δ P / Δ t \vert}{{σ}_{P}}} {e}^{\frac{- \sqrt{2} \vert Δ S / Δ t \vert}{{σ}_{S}}} \qquad Eq . 6$$

In this equation Sₙ is the state at time index n, is the state at the previous time index, ΔP and ΔS are the change in the parasympathetic and sympathetic values, respectively, between the previous state and the current state, Δt is the change in time in seconds between the time at index n−1 and the time at index n, and σₚ and us are the parameters that define the propensity of the parasympathetic and sympathetic values to change in a unit of time. To calculate the probability of observing a particular heart rate given an “actual” heart rate computed as described above, we assume a Gaussian distribution having a mean equal to the mean observed heart rate at the particular time and standard deviation equal to the standard deviation of instantaneous heart rates in a short (approximately 5 seconds) window of the preceding values of instantaneous heart rate. The probability of observing the heart rate is then given by:

$$P ( {HR}_{n} \vert {S}_{n} ) = \frac{1}{\sqrt{2 π {σ}_{n}^{2}}} {e}^{- {( {HR}_{n}^{calc} - {μ}_{n} )}^{2} / ( 2 {σ}_{n}^{2} )} \qquad Eq . 7$$

In this equation, HRₙ is the observed heart rate at time corresponding to index n, σₙ and μₙ, are the standard deviation and mean at time n, estimated as explained above, and HRₙ^{calc} is the “true” or “calculated” heart rate at time n. Finally, it should be noted that time must be discretized into points with a desirable resolution (such as 0.5 or 1 second between samples) and the sympathetic and parasympathetic values must also be on a predefined, quantized range. To obtain the maximum a posteriori estimate in a reasonable amount of time, the Viterbi algorithm, as defined in the communication theory literature is employed. As of now, the parasympathetic and sympathetic values obtained using this approach do not exactly match expected values, but the theory is sound and further experimentation with the parameters and guiding functions is expected to yield improved results. In another embodiment, the particular equations listed may be altered in particular form, while maintaining the main anatomical features discussed above.

In yet another embodiment, an integrate and fire model of heart beat generation is used to construct expected RR interval probability distribution functions, which are compared against an empirical RR interval histogram constructed from approximately five minutes of data or more in order to determine sympathetic and parasympathetic activity. The integrate and fire model can be described by the following equation:

$$1 = {\int }_{{t}_{k}}^{{t}_{k + 1}} s ( t ) - p ( t ) + c d t \qquad Eq . 8$$

In this equation, s (t) is a Poisson process describing sympathetic activity, where each event has amplitude A_{S} and the events occur with an average rate λₛ, p(t) is a Poisson process describing parasympathetic activity, where each event has amplitude Aₚ and an average rate. λₚ. c is a constant that determines the intrinsic average heart rate in the absence of autonomic function. tₖ and tₖ₊ₗ are the times corresponding to the current heartbeat and the next consecutive heartbeat. In essence, once the integral of the constant plus the positive contributions of the sympathetic system and the negative contributions of the parasympathetic system surpass the threshold of 1, a heart beat occurs. The goal of this approach is to generate inter-beat interval probability distribution functions based on said model that correspond to various values of sympathetic and parasympathetic activity, as defined by the free parameters λₛ, Aₛ, λₚ, and Aₚ, and then to find which set of parameters results in the best match between the calculated probability density function and the actual observed inter beat interval histogram from the physiological recording. In one embodiment, this would involve constructing a family of distributions through numerical simulation experiments with the given model and simply comparing some error function between the computed and observed distribution functions. In another embodiment, the analytic form of the expected distribution, parameterized by λₛ, Aₛ, λₚ, Aₚ, and c, is calculated and the parameter values are assigned by comparison to physiologic data. In one embodiment, the value for the constant c can be determined empirically from double pharmacologic blockade experiments, in which heart beat events are recorded in subjects following the administration of drugs that inactivate both the parasympathetic and the sympathetic nervous activity. In yet another embodiment, we assume that contributions from the sympathetic system are negligible due to sympathetic blockage by drugs or by post-processing of the heart beat signal by a process such as but not limited to subtraction of linearly predicted values, so the analytical form of the probability distribution function for this case is represented by the equation:

$$P ( k = 0 ) = {e}^{λ \frac{1}{c}} \\ P ( k = {k}_{0} ) = {{e}^{- λ ( \frac{1 + {k}_{0} {A}_{p}}{c} )} ( \frac{{λ}_{p}}{c} )}^{{k}_{0}} {( {A}_{p} )}^{{k}_{0} - 1} \frac{1}{{k}_{0} !} {( \frac{1}{{A}_{p}} + {k}_{0} )}^{{k}_{0} - 1} for k = 1 , 2 , 3 … \qquad Eq . 9$$

In this equation, k is any integer and k₀ is the particular value that k takes on. The parameters c, λₚ, and Aₚ are as defined above. This distribution can be fit to the RR interval histogram recorded from a patient under sympathetic blockade conditions in any way familiar to practitioners of the art (for example, by nonlinear least squares minimization, comparison to a family of functions generated with all possible parameter combinations on a particular range, or computation of higher order statistics like the mean, standard deviation, kurtosis, etc.). The above procedures would produce an estimate of parasympathetic tone.

In yet another embodiment, the sympathetic tone is be estimated using the value of parasympathetic tone calculated as in any of the above embodiments and instantaneous heart rate via the equation HR(t)=HR₀(1+s(t) p(t)), where HR(t) is the heart rate at time t, HR₀ is the baseline heart rate in the absence of autonomic nervous function, s(t) is the unknown value of sympathetic activity at time t, and p(t) is the estimated value of parasympathetic activity at time t. In another embodiment, the function HR(t)=HR₀+k₁s(t)−k_{2P}(t) may be used, where k₁ and k₂ are some constant weighting factors. In yet another embodiment, a nonlinear function of s(t) and p(t) may be used. In yet another embodiment, the sympathetic activity at a particular time may be known, and the parasympathetic activity may be unknown, in which case the above equations can also be solved to produce the unknown quantity.

In yet another embodiment, a more physiologically-motivated mathematical model of the sinoatrial node is used to predict the inter-beat interval distribution for various levels of sympathetic and parasympathetic activity. The basic sinoatrial node model has been described by Demir et al. [8], and would need to be modified to include sympathetic and parasympathetic inputs. In one embodiment, the sympathetic contribution is in the form of an inward, depolarizing current. The magnitude of this current is computed by convolving a Poisson process describing sympathetic activity by the sympathetic nervous activity-to-heart rate transfer function measured and described by Berger et al. [9]. In this embodiment, the sympathetic Poisson process is parameterized by a mean rate and can be generated by computer simulation. In another embodiment, the form of the sympathetic contribution is that of a change in the sodium channel conductance as well as release of intracellular calcium ions, also calculated based on the measurements of Berger et al. and the model of Demir et al. In yet another embodiment, the parasympathetic contribution is in the form of an outward, hyperpolarizing current. The magnitude of this current is computed by convolving a Poisson process describing parasympathetic activity by the parasympathetic nervous activity-to-heart rate transfer function as described by Berger et al. In this embodiment, the parasympathetic activity Poisson Process is parameterized by a parasympathetic average rate and is generated by computer simulation. In another embodiment, the form of the parasympathetic contribution is that of a change in the sodium and potassium channel conductances, and removal or sequestering of the intracellular calcium stores, as can be determined based on the combined works of Demir et al. and Berger et al.

In another embodiment, this mathematical model is further utilized to simulate sinoatrial node activity, and from this, the heart beat events are determined. These heart beat events are further analyzed to yield a library of unique probability distribution functions of RR intervals for various values of sympathetic and parasympathetic activity on a predefined range, such as but not limited to 0 Hz to 10 Hz for each. In another embodiment, the RR interval histogram obtained from at least five minutes of inter heart beat interval data is compared to this entire library of model distributions and the best match is chosen as the representation of actual parasympathetic and sympathetic nervous activity. In yet another embodiment, the library of model distributions can be stored in the form of a generalized equation that describes the distributions, rather than storing each distribution separately.

In yet another embodiment, the above model can be expanded to include spread of depolarization across a three-dimensional computational model of the heart. This signal can then be processed to yield the expected electrocardiogram signal. The simulated electrocardiogram signal can then be processed to understand whether sympathetic or parasympathetic activity can be measured directly from fine fluctuations in the electrocardiogram waveform. This method would allow the direct, fine time resolution measurement of the ANS parameters of interest.

In yet another embodiment, the sympathetic and parasympathetic tone values obtained as explained in any of the above embodiments are further processed to yield an estimate of physiological stress. In one embodiment, this can be done by taking the ratio of sympathetic to parasympathetic activity: Stress=S/P, where S is the value of sympathetic activity and P is the value of parasympathetic activity. Given the definition of stress as the dominance of sympathetic over the parasympathetic branches, higher values of this ratio would correspond to more stressful states. In another embodiment, stress can be quantified as the difference between sympathetic and parasympathetic activity: Stress=S-P. This definition also implies that sympathetically dominant states would give higher values for the calculated stress. In yet another embodiment, the S and P values in the above can be modified in a linear way: Stress=aS−bP+c, where a, b, and c are scaling constants determined empirically from patient data in control and pharmacologic blockade conditions. For example, S and P values obtained under control conditions (sitting, relaxed patient) can be assigned to correspond to a Stress value of 0, S and P values obtained under parasympathetic blockade can be assigned a Stress value of 1, and S and P values obtained under sympathetic blockade can be assigned a Stress value of −1. These three equations can be solved to yield the correct values of a, b, and c. In this embodiment, the a, b, and c values would be obtained from a group of patients and averaged to determine the best set of parameters to use on any successive subject without having to first go through this calibration step.

In yet another embodiment, any combination of one or more of the above signals (including signals derived from the intervals between heart beats as well as the instantaneous lung volume itself or other signals which reflect respiratory activity, the arterial blood pressure signal itself or other signals which reflect cardiovascular activity, and the like) are detected and processed to estimate stress or used to reduce the noise of the stress estimate. CSI methods described above may are well adapted to process multiple physiologic signals such as these and in this preferred embodiment may be applied to obtain an estimate of physiologic stress.

In another preferred embodiment the estimate of physiologic stress is used to determine whether a subject is lying (deceitful) or truthful when speaking spontaneously or when answering one or more questions.

In another preferred embodiment the estimate of physiologic stress is used to monitor the status of a patient with a medical condition which would benefit from such monitoring. Such a patient might be in an intensive care or critical care unit, undergoing exercise testing, or be an ambulatory patient with remote monitoring of his/her physiologic signals.

It is recognized that modifications and variations of the present invention will occur to those skilled in the art and it is intended that all such modifications and variations be included within the scope of the present invention.

### REFERENCES

Sarbach et al., Biological marker for stress states, U.S. Pat. No. 7,049,149
1. Akselrod, S., et al., Power spectrum analysis of heart rate fluctuation: a quantitative probe of beat-to-beat cardiovascular control. Science, 1981 213(4504): p. 220-2.
2. Xiao, X., et al., Effects of simulated microgravity on closed-loop cardiovascular regulation and orthostatic intolerance: analysis by means of system identification. J Appl Physiol, 2004. 96(2): p. 489-97.
3. Mukkamala, R., K. Toska, and R. J. Cohen, Noninvasive identification of the total peripheral resistance baroreflex. Am J Physiol Heart Circ Physiol, 2003. 284(3): p. H947-59.
4. Mukkamala, R., et al., System identification of closed-loop cardiovascular control mechanisms: diabetic autonomic neuropathy. Am J Physiol, 1999. 276(3 Pt 2): p. R905-12.
5. Mullen, T. J., et al., System identification of closed-loop cardiovascular control: effects of posture and autonomic blockade. Am J Physiol, 1997. 272(1 Pt 2): p. H448-61.
6. Triedman, J. K., et al., Respiratory sinus arrhythmia. time domain characterization using autoregressive moving average analysis. Am J Physiol, 1995. 268(6 Pt 2): p. H2232-8.
7. Xiao, X., Principal Component Based System Identification and Its Application to the Study of Cardiovascular Regulation, in Health Sciences and Technology. 2004, MIT: Cambridge. p. 212.
8. Demir, S. S., et al., A mathematical model of a rabbit sinoatrial node cell. Am J Physiol, 1994. 266(3 Pt 1): p. C832-52.
9. Berger, R. D., J. P. Saul, and R. J. Cohen, Transfer function analysis of autonomic regulation. I. Canine atrial rate response. Am J Physiol, 1989. 256(1 Pt 2): p. H142-52.

What is claimed is:

1. Method for determining physiological stress comprising:
detecting one or more physiologic signals in a subject;
processing the one or more physiologic signals to obtain one or more processed signals; and
estimating from the one or more processed signals the subject's stress level.

2. The method of claim 1 wherein the one or more physiological signals includes the electrocardiogram, or the instantaneous lung volume or other signal reflecting respiratory activity, or the arterial blood pressure or other signal reflecting cardiovascular activity.

3. The method of claim 1 wherein the one or more processed signals includes the intervals between heart beats or the instantaneous heart rate.

4. The method of claim 1 wherein the processing step further includes computing the instantaneous rate of change, or the first difference, or the absolute value of the first difference, or the second difference, or the absolute value of the second difference.

5. The method of claim 1 further comprising computing shot noise, or a measure of spectral curvature or a correlation coefficient.

6. The method of claim 1 wherein the processing is conducted in real time.

7. The method of claim 1 wherein the processing is evaluated in a time window on the order of 1 to 10 seconds, or a time window of approximately 20 seconds, or a longer time window of one or more minutes.

8. The method of claim 1 further comprising computing the magnitude of sympathetic and parasympathetic activity.

9. The method of claim 8 wherein physiological stress is determined from a function of the sympathetic and parasympathetic activity.

10. The method of claim 8 wherein stress is estimated by computing S/P wherein S is sympathetic activity and P is parasympathetic activity.

11. The method of claim 8 wherein the stress is estimated by computing aS−bP+c wherein S and P are sympathetic and parasympathetic activity respectively and a, b and c are constants.

12. The method of claim 1 further comprising the estimation of a transfer function.

13. The method of claim 1 further comprising the application of cardiovascular system identification (CSI).

14. The method of claim 1 further comprising computing a time-series of state vectors satisfying a Hidden Markov Model representation of hidden physiologic states and observable physiologic signals.

15. The method of claim 1 further comprising estimating Poisson process parameters.

16. The method of claim 1 further comprising the application of an integrate and fire model of the sino-atrial node.

17. The method of claim 8 wherein the magnitude of sympathetic and parasympathetic activity is computed by extrapolating short-window estimates to the “infinite data set”.

18. The method of claim 1 further comprising using the estimate of physiologic stress to determine whether a subject is lying (deceitful) or truthful when speaking spontaneously or when answering one or more questions.

19. The method of claim 1 wherein the estimate of physiologic stress is used to monitor the status of a patient.

20. Apparatus for determining physiological stress comprising:
means for detecting one or more physiologic signals in a subject;
processing means for processing the one or more physiologic signals to obtain one or more processed signals; and
means for estimating from the one or more processed signals the subject's stress level.

21. The apparatus of claim 20 further comprising means for using the estimate of physiologic stress to determine whether a subject is lying (deceitful) or truthful when speaking spontaneously or when answering one or more questions.

22. The apparatus of claim 20 further including means for using the estimate of physiological stress to monitor the status of a patient.
